(19) United States
(12) Patent Application Publication
(10) Pub. No.: US 2022/0227843 A1
(43) Pub. Date: Jul. 21, 2022

(54) CORONAVIRUS-BINDING MOLECULES AND METHODS OF USE THEREOF

(71) Applicant: Antaimmu BioMed Co., Ltd., Hsinchu City (TW)

(72) Inventors: San-Tai Shen, Hsinchu City (TW); Ing-Chien Chen, Hsinchu City (TW); Kuang-Kai Liu, Hsinchu City (TW); Frederick Y. Luh, Hsinchu City (TW)

(73) Assignee: Antaimmu BioMed Co., Ltd., Hsinchu City (TW)

(21) Appl. No.: 17/573,657

(22) Filed: Jan. 12, 2022

Related U.S. Application Data
(60) Provisional application No. 63/137,738, filed on Jan. 15, 2021.

(51) Int. Cl. C07K 16/10; C12N 7/00

(52) U.S. Cl. CPC C07K 16/10; C12N 7/00; C07K 2317/76; C12N 2770/20011; C07K 2317/92; C07K 2317/622; C07K 2317/565

(57) ABSTRACT

The present invention provides binding molecules, including monoclonal antibodies, multi-specific antibodies, and antibody fragments, that specifically bind to the coronavirus, such as SARS-CoV-2, and methods of use thereof. In some aspects of the invention, the binding molecules are human antibodies, fragments, or derivatives thereof that specifically bind to SARS-CoV-2 spike protein. In some aspects of the invention, the binding molecules function to neutralize SARS-CoV-2. The present invention also relates to methods of using the binding molecules and compositions for diagnosis and treatment.

### CROSS REFERENCE TO RELATED APPLICATIONS

This application claims priority to provisional Application No. 63/137,738, filed on Jan. 15, 2021. The contents thereof are included herein by reference.

### BACKGROUND OF THE INVENTION

### 1. Field of the Invention

The present invention relates to binding molecules, such as antibodies or the fragments thereof, which bind to coronaviruses as well as the methods of use thereof. In particular, the present invention is directed to binding molecules, such as an IgG or a scFv, which bind to coronaviruses, a method for decreasing S protein-mediated SARS-CoV-2 binding to cells as well as a method for treating, preventing, or alleviating the symptoms of a coronavirus-mediated disorder in a subject in need.

### 2. Description of the Prior Art

Human coronaviruses, first identified in the mid-1960s, are a large family of viruses that cause illness ranging from the common cold to more severe diseases such as Middle East Respiratory Syndrome (MERS-CoV), which was first reported in Saudi Arabia in 2012, Severe Acute Respiratory Syndrome (SARS-CoV), which was first recognized in China in 2002, and 2019 coronavirus disease (COVID-19), which was first reported from Wuhan, China, in 2019.

Coronaviruses are enveloped positive-sense RNA viruses. The most prominent feature of coronaviruses is the club-shaped spike projecting from the surface of the virion. The size of the genome of coronaviruses ranges between approximately 26,000 and 32,000 bases, including a variable number of open reading frames (ORFs). The first ORF encodes non-structural proteins (nsps), while the remaining ORFs encode accessory proteins and structural proteins. Coronavirus virus particles contain four main structural proteins. These are the spike (S), membrane (M), envelope (E), and nucleocapsid (N) proteins, all of which are encoded within the 3′ end of the viral genome. The spike surface glycoprotein plays an essential role in binding to receptors on the host cell

Since the outbreak of COVID-19 in China in the end of 2019, COVID-19 has been rapidly spread globally and led to a global public health crisis. Subsequently, the World Health Organization (WHO) declared a global pandemic on the 21 Mar. 2020.

COVID-19 is caused by severe acute respiratory syndrome coronavirus 2 (SARS-CoV-2). SARS-CoV-2 binds to the host cell through spike protein (S protein). The spike protein includes an extracellular N-terminus, a transmembrane (TM) domain anchored in the viral membrane, and a short intracellular C-terminal segment The amino acid sequence of SARS-CoV-2 S protein is shown in SEQ ID NO:1. The spike protein is a potential target for developing neutralizing antibody and therapeutic methods.

As of the end of 2020, tens of millions of COVID-19 cases had been confirmed around the world. There is an urgent need for preventive and antiviral therapies for COVID-19 control. Antibodies that specifically bind to SARS-CoV-2 with high affinity and/or neutralizing ability could be important in the detection, prevention, and treatment of COVID-19 infection.

### SUMMARY OF THE INVENTION

The present invention provides a binding molecule, such as an antibody, a recombinant antibody, a monoclonal antibody, an antibody derivative or the fragment thereof, wherein the binding molecule specifically binds to coronavirus, such as SARS-CoV-2. The binding molecule specifically binds to S protein of coronavirus, such as S protein of SARS-CoV-2 (SEQ ID NO:1), S protein of SARS CoV (SEQ ID NO:2), and S protein of MERS CoV (SEQ ID NO:3). In certain aspects, the binding molecule specifically binds to the spike protein of SARS-CoV-2, and function to neutralize SARS-CoV-2.

In some embodiments, the binding molecule is a multispecific binding molecule, such as a multispecific antibody or a multispecific antibody fragment. In some embodiments, the binding molecule is a multispecific binding molecule including more than two binding domains specifically binding to different epitopes on S protein.

In certain aspects, the disclosure provides a binding molecule that specifically binds to SARS-CoV-2 S protein including VL and VH domains that are at least 80%, 90% or 100% identical in amino acid sequence to the VL and VH domains, respectively, of an antibody selected from the group consisting of:

antibodies ECD-1, ECD-2, ECD-3, ECD-5, ECD-10, ECD-11, ECD-12, ECD-14, ECD-21, ECD-22, ECD-24, ECD-26, ECD-28, ECD-30, ECD-35, ECD-36, ECD-37, ECD-39, ECD-45, ECD-49, and RBD-21. The VL domains of antibodies ECD-2, ECD-14, ECD-21, ECD-28, and ECD-36 include amino acid sequences of SEQ ID NO: 4, 6, 8, 10, and 12 respectively. The VH domains of antibodies ECD-2, ECD-14, ECD-21, ECD-28, and ECD-36 include amino acid sequences of SEQ ID NO: 5, 7, 9, 11, and 13 respectively.

In some embodiments, the monoclonal antibody is a human IgG, IgM, IgE, IgA, or IgD molecule. In some embodiments, the SARS-CoV-2 S binding molecule is an IgG1, IgG2, IgG3, or IgG4 subclass. Optionally, the binding molecule is an IgG1 or IgG4 antibody.

In certain aspects, the disclosure provides a binding molecule that specifically binds to SARS-CoV-2 S protein. The binding molecule includes:

(a) a light chain including a light chain CDR1, a light chain CDR2 and a light chain CDR3 (LCDR 1, LCDR2, and LCDR3) that are identical in amino acid sequence to the LCDR 1, LCDR2, and LCDR3 of an antibody selected from the group consisting of: antibodies ECD-2, ECD-14, ECD-21, ECD-28, and ECD-36;
(b) a heavy chain including a heavy chain CDR1, a heavy chain CDR2 and a heavy chain CDR3 (HCDR 1, HCDR2, and HCDR3) that are identical in amino acid sequence to the HCDR 1, HCDR2, and HCDR3 of an antibody selected from the group consisting of: antibodies ECD-2, ECD-14, ECD-21, ECD-28, and ECD-36; or
(c) a light chain including LCDR 1, LCDR2, and LCDR3 and a heavy chain including HCDR 1, HCDR2, and HCDR3 that are identical in amino acid sequence to an antibody selected from the group consisting of:
antibodies ECD-2, ECD-14, ECD-21, ECD-28, and ECD-36;
wherein the LCDR 1, LCDR2, and LCDR3 of antibody ECD-2 separately includes the amino acid sequences of SEQ ID NO: 14, 15, and 16; the LCDR 1, LCDR2, and LCDR3 of antibody ECD-14 separately includes the amino acid sequences of SEQ ID NO: 17, 18, and 19; the LCDR 1, LCDR2, and LCDR3 of antibody ECD-21 separately includes the amino acid sequences of SEQ ID NO: 20, 21, and 22; the LCDR 1, LCDR2, and LCDR3 of antibody ECD-28 separately includes the amino acid sequences of SEQ ID NO: 23, 24, and 25; the LCDR 1, LCDR2, and LCDR3 of antibody ECD-36 separately includes the amino acid sequences of SEQ ID NO: 26, 27, and 28, and
wherein the HCDR 1, HCDR2, and HCDR3 of antibody ECD-2 separately includes the amino acid sequences of SEQ ID NO: 30, 31, and 32; the HCDR 1, HCDR2, and HCDR3 of antibody ECD-14 separately includes the amino acid sequences of SEQ ID NO: 33, 34, and 35; the HCDR 1, HCDR2, and HCDR3 of antibody ECD-21 separately includes the amino acid sequences of SEQ ID NO: 36, 37, and 38; the HCDR 1, HCDR2, and HCDR3 of antibody ECD-28 separately includes the amino acid sequences of SEQ ID NO: 39, 40, and 41; the HCDR 1, HCDR2, and HCDR3 of antibody ECD-36 separately includes the amino acid sequences of SEQ ID NO: 42, 43, and 44.

In certain aspects, the binding molecule includes amino acid sequences identical to the FR1, FR2, FR3 and FR4 of the heavy chain variable domain and the light chain variable domain of the antibody selected from the group consisting of: antibodies ECD-2 (SEQ ID NO:4 and 5), ECD-14 (SEQ ID NO: 6 and 7), ECD-21 (SEQ ID NO: 8 and 9), ECD-28 (SEQ ID NO:10 and 11), and ECD-36 (SEQ ID NO:12 and 13).

In some embodiments, the binding molecule includes a light chain constant domain wherein the amino acid sequence is SEQ ID NO: 29.

In some embodiments, the binding molecule includes a heavy chain constant domain wherein the amino acid sequence is SEQ ID NO: 45.

In another aspect of the present invention, the disclosure provides a composition including at least one neutralizing binding molecule that specifically binds to a region of coronavirus S protein, wherein the binding molecule neutralizes the virus at an IC₅₀ of 1 μg/ml or less. In some embodiments, the IC₅₀ of neutralizing antibodies is less than 0.77 μg/ml, or less than 0.4 μg/ml, or preferably less than 0.3 μg/ml.

In another aspect, the present invention provides a pharmaceutical composition including one or more binding molecule selected from the binding molecules described above. In some embodiments the composition further includes one or more antibodies specifically binding to a SARS-CoV-2, or anti-viral agents.

In some embodiments, the invention provides an isolated nucleic acid molecule including a nucleotide sequence that encodes binding molecule according to any one of the preceding embodiments. In certain aspects, the disclosure provides a vector including the nucleic acid molecule according to any one of the preceding embodiments.

In certain aspects, the disclosure provides a host cell including a vector according to any one of the preceding embodiments or a nucleic acid molecule according to any one of the preceding embodiments.

In certain aspects, the invention provides a host cell that produces the binding molecule or fragment thereof according to any one of the preceding embodiments.

In certain aspects, the disclosure provides a method for decreasing S protein-mediated SARS-CoV-2 binding to cells. The method includes the step of contacting the SARS-CoV-2 with a binding molecule according to any one of the preceding embodiments. In certain embodiments, the cells express angiotensin converting enzyme 2 (ACE2).

In certain aspects, the invention provides a method for decreasing the SARS-CoV-2 viral load in a subject in need thereof and including the step of administering a binding molecule according to any one of the preceding embodiments.

In certain aspects, the disclosure provides a method for treating, preventing or alleviating the symptoms of a coronavirus-mediated disorder in a subject in need thereof. The coronavirus-mediated disorder includes but not limit to SARS CoV, MERS CoV and SARS-CoV-2 mediated disorder. The method includes the step of administering to the subject one or more binding molecule or pharmaceutical compositions according to any one of the preceding embodiments or a composition according to any one of the preceding embodiments. In certain embodiments, the coronavirus-mediated disorder is COVID-19.

In certain aspects, the disclosure provides a method for treating, preventing or alleviating the symptoms of a coronavirus-mediated disorder in a subject in need thereof. The coronavirus-mediated disorder includes but not limit to SARS CoV, MERS CoV and SARS-CoV-2 mediated disorder. The method includes the step of administering to the subject one or more binding molecule according to any one of the preceding embodiments, further including at least one additional therapeutic agent including but not limit to: one or more antibodies that specifically bind to coronavirus, and/or one or more anti-viral agent.

In certain aspects, the disclosure provides a method for detecting SARS-CoV-2. The method includes the steps of:

(a) contacting the binding molecule of the present invention to a sample or specimen derived from an individual suspected to be infected by SARS-CoV-2; and
(b) detecting the presence of the binding molecule.

The invention contemplates combinations of any of the foregoing aspects and embodiments of the invention.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 shows the results of single colony ELISA analyzing the binding of S protein scFvs against S protein ECD, and S protein RBD domains that expressed by E. coli.

FIG. 2A and FIG. 2B show the results of ELISA analyzing the binding of S protein IgGs at different concentrations against S protein expressed by HEK293 cell.

FIG. 3 shows the results of cell binding analysis of S protein IgGs against S protein expressed on CHO cell surface.

FIG. 4 shows the ACE2 competition assay of S protein IgGs.

FIG. 5 shows the binding affinities of the tested antibodies toward S protein RBD of B.1.351 strain.

FIG. 6 shows the pseudovirus neutralization rate of the tested antibodies.

### DETAILED DESCRIPTION

Provided herein are binding molecules which exhibit specific binding to coronavirus. In certain aspects, the binding molecules are antibodies or the fragments thereof specifically bind to the spike protein of SARS-CoV-2. In certain aspects, the binding molecules are able to neutralize SARS-CoV-2.

### Definition

Terms are defined herein for clarity, and the inclusion of such definitions herein should not necessarily be construed to represent a substantial difference over what is generally understood in the art.

The term “binding molecule” used herein covers different kinds of molecules able to specifically bind to a target molecule, or molecules including at least one antigen-binding domain, including but are not limited to monoclonal antibodies, recombinant antibodies, multispecific antibodies, antibody derivatives and antibody fragments. As used herein, the terms “specific binding,” and “specifically bind to” refer to the non-covalent interactions of the type which occur between a binding molecule and a target or antigen for which the binding is specific. The strength, or affinity of binding molecule can be expressed in terms of the dissociation equilibrium constant (K_{D}) of the interaction, wherein a smaller K_{D} represents a greater affinity. A binding molecule of the present invention is said to specifically bind to a coronavirus, including SARS-CoV-2, SARS CoV, and MERS CoV epitope when the K_{D} is ≤1 μM, preferably ≤100 nM, more preferably ≤10 nM, and most preferably ≤200 pM to about 1 pM, as measured by assays such as radioligand binding assays or similar assays known to those skilled in the art. The term “epitope” includes any protein determinant capable of specific binding to an immunoglobulin, a scFv, or a T-cell receptor.

The term “antibody” as used herein, is intended to refer to immunoglobulin molecules comprised of four polypeptide chains: two heavy (H) chains and two light (L) chains inter-connected by disulfide bonds (i.e., “full antibody molecules”, such as IgG), as well as multimers thereof (e.g. IgM). Each heavy chain includes a heavy chain variable region (“HCVR” or “VH”) and a heavy chain constant region. Each light chain is comprised of a light chain variable region (“LCVR” or “VL”) and a light chain constant region. The HCVR and LCVR can be further subdivided into regions of hyper variability, termed complementarity determining regions (CDR), interspersed with regions that are more conserved, termed framework regions (FR). Each HCVR and LCVR is composed of, arranged from amino-terminus to carboxy-terminus, FR1, CDR1, FR2, CDR2, FR3, CDR3, and FR4. In certain embodiments of this invention, the FRs of the antibody (or antigen binding fragment thereof) may be identical to the human germline sequences or may be naturally or artificially modified.

The term “recombinant antibody,” as used herein, refers to antibodies that are prepared, expressed, created, or isolated by recombinant means, such as antibodies expressed using a recombinant expression vector transfected into a host cell, antibodies isolated from a recombinant, combinatorial antibody library, antibodies isolated from an animal (e.g., a mouse) that is transgenic for human immunoglobulin genes or antibodies prepared, expressed, created, or isolated by any other means that involves splicing of human immunoglobulin gene sequences to other DNA sequences. Such recombinant antibodies may include humanized, CDR grafted, chimeric, in vitro generated (e.g., by phage display) antibodies, binding molecules, and may optionally include constant regions derived from human germline immunoglobulin sequences. Also, “recombinant antibody” may direct to a portion of an intact antibody, including, without limitation, Fv, Fab, Fab′, F(ab′) 2, diabodies, scFv, and single domain antibodies (e.g., variable heavy domain (VHH)).

The term “antibody fragment” or “antigen binding fragment” used herein directs to a portion of an intact antibody, including but are not limited to Fv, Fab, Fab′, F(ab′)2, diabodies, single-chain antibody molecules (e.g. scFv), and single domain antibodies such as VHH.

The terms “CoV-S”, “S protein” or “spike protein” as used herein refer to spike protein of coronavirus. S protein normally exists in a metastable, prefusion conformation; once the virus interacts with the host cell, extensive structural rearrangement of the S protein occurs, allowing the virus to fuse with the host cell membrane. The total length of SARS-CoV-2 S protein is 1273 amino acids (aa) and consists of a signal peptide (residues 1-13) located at the N-terminus, the S1 subunit (residues 14-685), and the S2 subunit (residues 686-1273). In the S1 subunit, there is an N-terminal domain (NTD, residues 14-305) and a receptor-binding domain (RBD, residues 319-541). The S2 domain includes the fusion peptide (FP, residues 788-806), heptapeptide repeat sequence 1 (HR1, residues 912-984), HR2 (residues 1163-1213), TM domain (residues 1213-1237), and the cytoplasm domain (residues 1237-1273).

As used herein, the term “S protein binding molecule” includes any binding molecule exhibiting specific binding to S protein of coronavirus. The term “S protein antibody”, “spike protein antibody”, “anti-S”, or “anti-spike” as used herein directs to antibody or the fragment thereof exhibits specific binding to S protein of coronavirus. The term “S scFv” or “S protein scFv” represents scFv able to bind S protein. The term “S IgG” or “S protein IgG” represents IgG able to bind S protein.

The terms “neutralizing binding molecule”, “neutralizing antibody” or “function to neutralize” as used herein means a binding molecule which can neutralize the virus at an IC₅₀ of 1 μg/ml or less. In some embodiments, the IC₅₀ of neutralizing antibodies is less than 0.77 μg/ml, or less than 0.4 μg/ml, preferably less than 0.3 μg/ml. In preferred embodiments, neutralizing antibodies are effective at antibody concentrations of less than 0.2 μg/ml. In the most preferred embodiments, neutralizing antibodies are effective at antibody concentrations of less than 0.1 μg/ml.

The term “nucleic acid molecule” as used herein refers to nucleic acid polymers encoding proteins of interest, such as the binding molecules including amino acid sequences in the present invention. The nucleic acid molecule sequence may be manufactured by genetic engineering techniques (e.g., a sequence encoding chimeric protein, a codon-optimized sequence, and/or an intron-less sequence), cloned into a vector, and introduced into a host cell, where it may reside as an episome or be integrated into the genome of the cell. A person skilled in the art can determine the sequences of a nucleic acid molecule according to the amino acid sequences intended to be encoded without undue experimentation, as well as the optimized codon corresponding with the host cell.

The term “vector” or “expression vector”, as used herein, refers to a nucleic acid molecule capable of carrying another nucleic acid to which it has been linked. In some embodiments, the vector is a plasmid, including a circular double stranded DNA into which additional DNA segments may be ligated. In some embodiments, the vector is a viral vector, wherein additional DNA segments may be ligated into the viral genome. In some embodiments, the vectors are capable of autonomous replication in a host cell into which they are introduced. In other embodiments, the vectors (e.g., non-episomal mammalian vectors) can be integrated into the genome of a host cell upon introduction into the host cell, and thereby are replicated along with the host genome.

The term “host cell”, as used herein, refers to a cell into which an expression vector has been introduced and also the progeny of the cell. Because certain modifications may occur in succeeding generations due to either mutation or environmental influences, such progeny may not, in fact, be identical to the progenitor cell, but are still included within the scope of the term “host cell” as used herein.

### Coronavirus S Protein Binding Molecule

The present invention provides coronavirus S protein binding molecules. In some embodiments, the binding molecule specifically binds to S protein of coronavirus, such as S protein of SARS-CoV-2 (SEQ ID NO:1), S protein of SARS CoV (SEQ ID NO:2), and/or S protein of MERS CoV (SEQ ID NO:3). In some embodiments, the binding molecule is an antibody or the fragment thereof. In some embodiments, the binding molecule is a multispecific binding molecule, such as a multispecific antibody or a multispecific antibody fragment. In some embodiments, the binding molecule is a heteroconjugated antibody composed of two covalently linked isomorphism antibodies. In some embodiments, the monoclonal antibody is a human IgG, IgM, IgE, IgA, or IgD molecule. In some embodiments, the SARS-CoV-2 S binding molecule is an IgG1, IgG2, IgG3, or IgG4 subclass. Alternatively, the binding molecule is a IgG1 or IgG4 antibody.

In certain aspects, the binding molecule includes VL and/or VH domains that are at least 80%, 90% or 100% identical in amino acid sequence to the VL and VH domains, respectively, of an antibody selected from the group consisting of: antibodies ECD-2, ECD-14, ECD-21, ECD-28, and ECD-36. The VL domains of ECD-2, ECD-14, ECD-21, ECD-28, and ECD-36 include amino acid sequences of SEQ ID NO: 4, 6, 8, 10, and 12 respectively. The VH domains of antibodies ECD-2, ECD-14, ECD-21, ECD-28, and ECD-36 include amino acid sequences of SEQ ID NO: 5, 7, 9, 11, and 13 respectively. In some embodiments, the amino acid sequences include more than one conservative amino acid substitutions.

In certain aspects, the disclosure provides a binding molecule that specifically binds SARS-CoV-2 S protein, wherein the binding molecule includes:

(a) a light chain including light chain CDR1, light chain CDR2 and light chain CDR3 (LCDR 1, LCDR2, and LCDR3) that are identical in amino acid sequence to the LCDR 1, LCDR2, and LCDR3 of an antibody selected from the group consisting of: antibodies ECD-2, ECD-14, ECD-21, ECD-28, and ECD-36;
(b) a heavy chain including heavy chain CDR1, heavy chain CDR2 and heavy chain CDR3 (HCDR 1, HCDR2, and HCDR3) that are identical in amino acid sequence to the HCDR 1, HCDR2, and HCDR3 of an antibody selected from the group consisting of: antibodies ECD-2, ECD-14, ECD-21, ECD-28, and ECD-36; or
(c) a light chain including LCDR 1, LCDR2, and LCDR3 and a heavy chain including HCDR 1, HCDR2, and HCDR3 that are identical in amino acid sequence to an antibody selected from the group consisting of:
antibodies ECD-2, ECD-14, ECD-21, ECD-28, and ECD-36;
wherein the LCDR 1, LCDR2, and LCDR3 of antibody ECD-2 separately includes the amino acid sequences of SEQ ID NO: 14, 15, and 16; the LCDR 1, LCDR2, and LCDR3 of antibody ECD-14 separately includes the amino acid sequences of SEQ ID NO: 17, 18, and 19; the LCDR 1, LCDR2, and LCDR3 of antibody ECD-21 separately includes the amino acid sequences of SEQ ID NO: 20, 21, and 22; the LCDR 1, LCDR2, and LCDR3 of antibody ECD-28 separately includes the amino acid sequences of SEQ ID NO: 23, 24, and 25; the LCDR 1, LCDR2, and LCDR3 of antibody ECD-36 separately includes the amino acid sequences of SEQ ID NO: 26, 27, and 28, and
wherein the HCDR 1, HCDR2, and HCDR3 of antibody ECD-2 separately includes the amino acid sequences of SEQ ID NO: 30, 31, and 32; the HCDR 1, HCDR2, and HCDR3 of antibody ECD-14 separately includes the amino acid sequences of SEQ ID NO: 33, 34, and 35; the HCDR 1, HCDR2, and HCDR3 of antibody ECD-21 separately includes the amino acid sequences of SEQ ID NO: 36, 37, and 38; the HCDR 1, HCDR2, and HCDR3 of antibody ECD-28 separately includes the amino acid sequences of SEQ ID NO: 39, 40, and 41; the HCDR 1, HCDR2, and HCDR3 of antibody ECD-36 separately includes the amino acid sequences of SEQ ID NO: 42, 43, and 44.

In some embodiments, the amino acid sequences have more than one conservative amino acid substitutions in the LCDR 1, LCDR2, and LCDR3 and HCDR 1, HCDR2, and HCDR3 region.

In some embodiments, the binding molecule includes amino acid sequences identical to the FR1, FR2, FR3 and FR4 of the antibody selected from the group consisting of: antibodies ECD-2, ECD-14, ECD-21, ECD-28 and ECD-36.

In some embodiments, the binding molecule includes a light chain constant domain wherein the amino acid sequence is SEQ ID NO:29. In some embodiments, the binding molecule includes a heavy chain constant domain wherein the amino acid sequence is SEQ ID NO: 45.

In some embodiments, the binding molecules include, but are not limited to, antibodies or antigen-binding portions which bind to (i) the 51 domain of SARS-CoV-2 S protein; (ii) the S2 domain of SARS-CoV-2 S protein; or (iii) both (i) and (ii). In some embodiments, the binding molecule binds to the NTD domain of 51. In some embodiments, the binding molecule binds to the RBD domain of 51. In some embodiments, the binding molecule is a multispecific antibody binds to both RBD and S2 domain.

In certain aspects, the binding molecule specifically binds to the spike protein of SARS-CoV-2, and performing to neutralize it.

In some embodiments, the binding molecule is a neutralizing binding molecule that specifically binds to a region of coronavirus S protein, wherein the binding molecule neutralizes the virus at an IC₅₀ of 1 μg/ml or less. In some embodiments, the IC₅₀ of neutralizing antibodies is less than 0.77 μg/ml, less than 0.4 μg/ml, preferably less than 0.3 μg/ml.

In some embodiments, the binding molecule is expressed by a nucleic acid vector including a nucleotide sequence that encodes binding molecule according to any one of the preceding embodiments. A person skilled in the art can determine the nucleotide sequence according to the amino acid sequences intended to be encoded without undue experimentation, as well in optimizing codon upon the character of the host cell.

In some embodiments, the vector encodes the heavy chain of the binding molecule of the invention or an antigen-binding portion thereof. In some embodiments, the vector encodes the light chain of the binding molecule or antigen-binding portion thereof. In some embodiments, the vector encodes a fusion protein, a modified antibody, an antibody fragment, and/or probes thereof. In some embodiments, the vectors are plasmids, retroviruses, adenoviruses, adeno-associated viruses (AAV), plant viruses such as cauliflower mosaic virus, tobacco mosaic virus, cosmids, YACs, EBV derived episomes, and the like.

The binding molecule is optionally further modified to enhance effectiveness. For example, the binding molecule includes Fc region, wherein the Fc is engineered using known method to enhance ADCC effect. In some embodiments, the binding molecule is conjugated to a cytotoxic agent such as toxoid from bacterial or fungus.

### Pharmaceutical Compositions

In one aspect, the present invention provides a pharmaceutical composition including the binding molecule described above. In some embodiments, the pharmaceutical composition further includes a pharmaceutical acceptable carrier, including solvent, dispersion media, coating, antibacterial and/or antifungal agent, isotonic and absorption delaying agent, and the analogous, compatible with pharmaceutical administration. For example, the composition further includes water, saline, ringer's solutions, dextrose solution, 5% human serum albumin, liposomes or non-aqueous vehicles.

In some embodiments, the pharmaceutical composition further includes therapeutic agents for the treatment of viral infection or inflammation such as nucleoside analogues, protease inhibitors, chemokine receptor antagonists, or interferon beta-lb. In some embodiments, the therapeutic agents are used to treat the symptoms of the SARS-CoV-2 infection and may be synergized with the effects of the binding molecule. Exemplary therapeutic agents include lopinavir-ritonavir, ribavirin, adalimumab, remdesivir, hydroxychloroquine, DAS181, lactoferrin, clevudine, tocilizumab, favipiravir, anti-SARS-CoV-2 convalescent plasma, recombinant human angiotensin-converting enzyme 2, aprotinin, clazakizumab, pamrevlumab, baricitinib, probiotic and combinations thereof.

### Method of Use

In one aspect, the present invention provides methods for detecting SARS-CoV-2 in a subject, including the steps of:

(a) contacting in vitro a biological sample from the subject with the binding molecule according to the present invention,
(b) detecting the presence of the binding molecule,
(c) determining the presence or the absence of SARS-CoV-2 spike protein in the biological sample.

In some embodiments, the methods for detecting SARS-CoV-2 spike protein include immunoassays such as ELISA, Western blot, tissue immunohistochemistry, and lateral flow assay.

In one aspect, the present invention provides methods for decreasing S protein-mediated coronavirus, such as SARS-CoV-2, binding to cells, including the step of contacting the SARS-CoV-2 with the binding molecule or a pharmaceutical composition according to the present invention. In one aspect, the present invention provides a method for treating, preventing, or alleviating the symptoms of a coronavirus-mediated disorder in a subject in need, including the step of administering to the subject the SARS-CoV-2 binding molecule or a pharmaceutical composition according to the present invention. Specifically, the coronavirus-mediated disorder is COVID-19.

In one aspect, the present invention provides methods for preventing SARS-CoV-2 related disease in a subject by administering the subject with the binding molecule of the present invention. For example, antibody ECD-1, ECD-2, ECD-3, ECD-5, ECD-10, ECD-11, ECD-12, ECD-14, ECD-21, ECD-22, ECD-24, ECD-26, ECD-28, ECD-30, ECD-35, ECD-36, ECD-37, ECD-39, ECD-45, ECD-49, or RBD-2 and any variants or the fragments thereof, may be administered in therapeutically effective amounts. Optionally, two or more anti-SARS-CoV-2 antibodies are co-administered. The binding molecules of the present invention can be administered by a variety of methods known in the art, although for many therapeutic applications, preferential route/mode of administration is subcutaneous, intramuscular, or intravenous infusion. In some embodiments, administration includes intraperitoneal, intrabronchial, transmucosal, intraspinal, intrasynovial, intraaortic, intranasal, ocular, otic, topical and buccal. Subjects at risk for SARS-CoV-2 related diseases include patients who have been exposed to the SARS-CoV-2. For example, the subjects have traveled to regions or to countries of the world in which other SARS-CoV-2 infections have been reported and confirmed. Administration of a prophylactic agent can occur prior to the manifestation of symptoms characteristic of the SARS-CoV-2 related disease, such that a disease is prevented or, alternatively, delayed in its progression.

Another aspect of the invention pertains to methods of treating a SARS-CoV-2 related disease or disorder in a patient. In some embodiments, the method involves administering the composition including the binding molecule according to the invention, or combination of agents that neutralize the SARS-CoV-2 to a patient suffering from the disease or disorder. In some embodiments, the invention provides methods for treating a SARS-CoV-2 related disease or disorder in a patient by administering an antibody of the present invention to a subject suffering from COVID-19. In some embodiments, the antibody is antibody ECD-2, ECD-14, ECD-21, ECD-28, or ECD-36 and any variants or the fragments thereof. Optionally, two or more anti-SARS-CoV-2 antibodies are co-administered. The method may include the step of co-administering the binding molecule of the invention and anti-viral agents, such as peptides, nucleic acids, small molecules, inhibitors, or RNAi.

The present invention will be further described by referring to the following examples, which do not limit the scope of the invention described in the claims.

### Example 1 Preparations of Antigens for Screening Antibodies

For S-protein expression, DNA sequence encoding ectodomain of SARS-CoV-2 S protein (positions 1 to 1211 of SEQ ID NO:1) was constructed into pcDNA3.4 vector. The plasmid DNA was transfected into HEK293 cell. The overexpressed SARS-CoV-2 S protein was harvested from the supernatant and purified with His-Trap affinity chromatography. The purity of the product was >95% as determined by SDS-PAGE.

### Example 2 Library Construction and Phage Display Screening

An scFv library was constructed on phagemid vector. Before the first round of panning, the library was titrated and more than 10⁹ clones were collected. Purified S proteins were coated on 96-Well plate, and then 10¹¹-10¹² CFU of PEG precipitated phage were add to each well of plate. The unbound phage was washed and the host E. coli was infected with bound phage. After two to three rounds of panning, single colony ELISA was assayed to confirm the binding. 308 (out of 384) phage strains able to bind SARS-CoV-2 S protein were obtained.

### Example 3 Single Colony ELISA

The obtained phages include S scFv phagemid vectors. The phages were infected into E. coli host, and then plating on LB-agar plate. Candidate colonies were picked up and grown in 2×YT plus 100 μg/ml ampicillin with rigorously shacking at 37° C. While OD₆₀₀>1, the cell culture was induced with IPTG to final concentration of 1 mM, and then incubated at 37° C. for overnight. After clarified by centrifuging at 4,000×g for 10 minutes, the secreted scFv present in the supernatant. Add secreted scFv onto Nunc-Immuno 96-Well plate coated with ECD(14-1211 residues) or RBD domain (319-541 residues) of SARS CoV2 S protein (SEQ ID NO:1). The signals were detected.

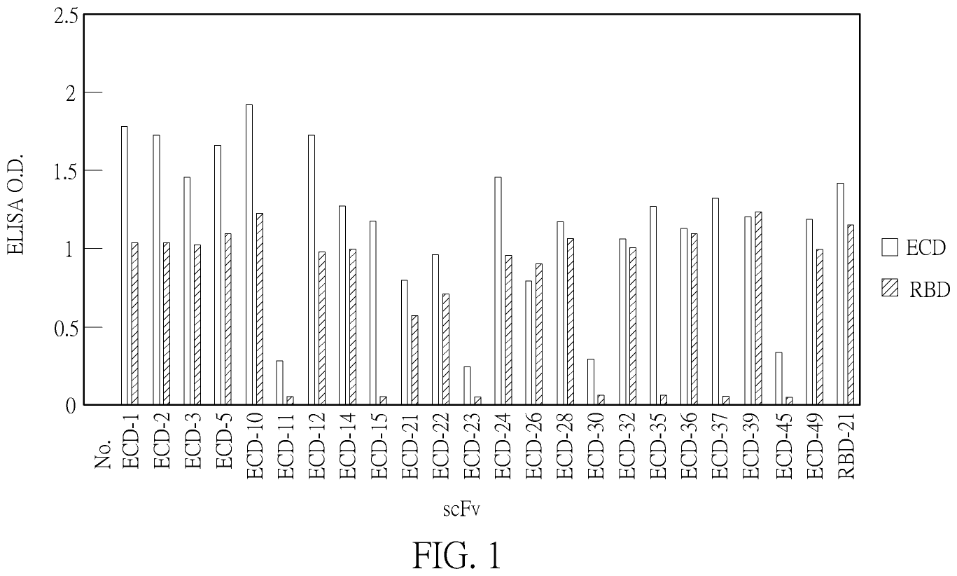

The results are shown in FIG. 1.

### Example 4 Transferring scFv to IgG Form

ECD-1, ECD-2, ECD-3, ECD-5, ECD-10, ECD-11, ECD-12, ECD-14, ECD-21, ECD-22, ECD-24, ECD-26, ECD-28, ECD-30, ECD-35, ECD-36, ECD-37, ECD-39, ECD-45, ECD-49, and RBD-21 scFvs were transfer to IgG form to increase stability and for further applications. For light chain IgG construction, 3 fragments of immunoglobulin light chain signal peptide, light chain variable domain and constant domain were PCR assembled, and then ligated into a first mammalian cell DNA vector to form a light chain plasmid. For heavy chain IgG construction, heavy chain signal peptide, variable domain and constant domain were ligated into a second mammalian cell DNA vector to form a heavy chain plasmid. IgG form antibodies were harvested from CHO cell co-transfected with both light and heavy chain plasmids.

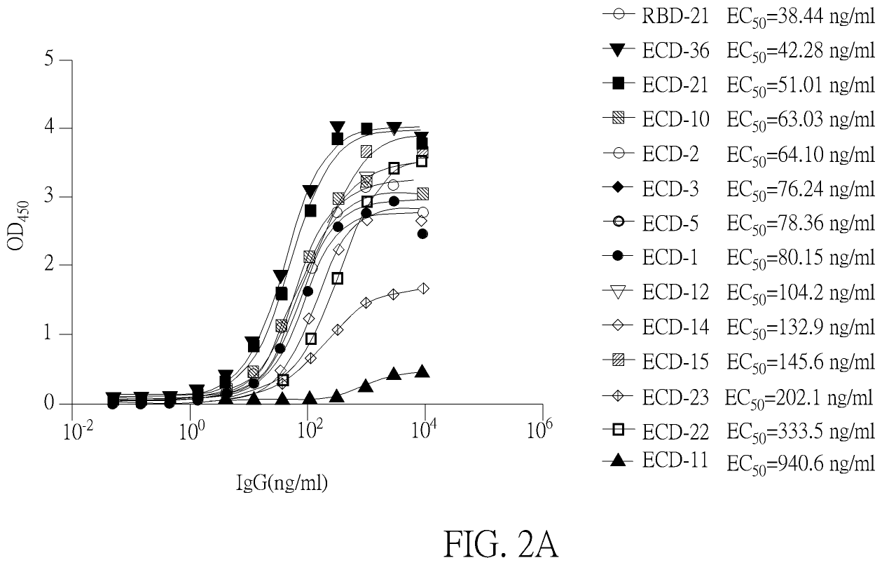

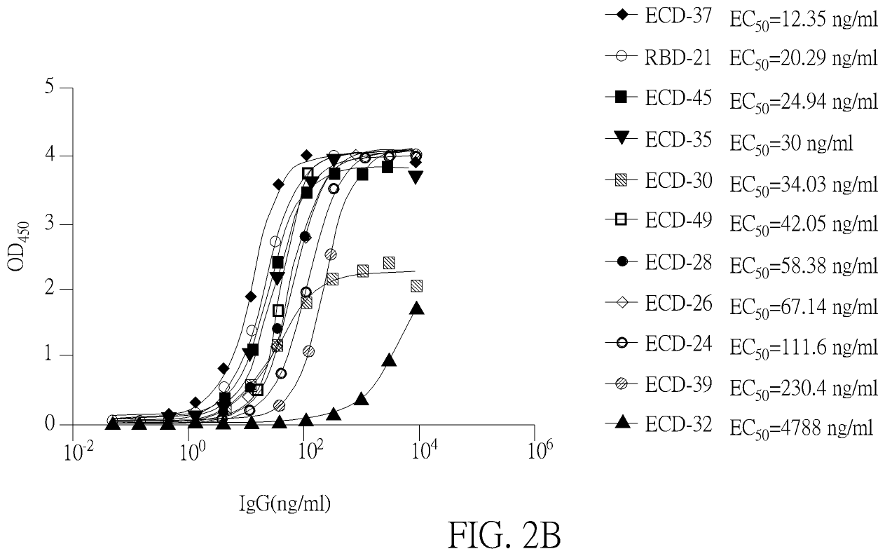

The binding affinities of ECD-1, ECD-2, ECD-3, ECD-5, ECD-10, ECD-11, ECD-12, ECD-14, ECD-21, ECD-22, ECD-24, ECD-26, ECD-28, ECD-30, ECD-35, ECD-36, ECD-37, ECD-39, ECD-45, ECD-49, and RBD-21 IgG toward S protein were examined by ELISA. S proteins purified from HEK293 cell were coated on ELISA plate at the concentration of 0.5 μg/well. After blocking with 5% skim milk, serial diluted IgG were added to each well. The signals were detected by anti-human antibody. The results are shown in FIG. 2A and in FIG. 2B.

The binding affinities of ECDs are shown in FIG. 2A and in FIG. 2B. As shown in FIG. 2A and in FIG. 2B, ECD-1, ECD-2, ECD-3, ECD-5, ECD-10, ECD-11, ECD-12, ECD-14, ECD-21, ECD-22, ECD-24, ECD-26, ECD-28, ECD-30, ECD-35, ECD-36, ECD-37, ECD-39, ECD-45, ECD-49, and RBD-21 IgG exhibit various affinities against S protein at various concentrations.

### Example 5 Epitope Competition

Purified S protein 0.5 μg/well were coated on ELISA plate. After blocking with 5% skim milk, 1 μg/well IgGs were add to each well. Five minutes later, 100 μl secreted scFv were added to each well. The scFv signals were detected with anti-c-myc antibody. The signals were low when epitopes of IgG and scFv were at the same place, and the signals were not altered when epitopes of IgG and scFv were different. ECD-1, ECD-2, ECD-3, ECD-5, ECD-10, ECD-11, ECD-12, ECD-14, ECD-21, ECD-22, ECD-24, ECD-26, ECD-28, ECD-30, ECD-35, ECD-36, ECD-37, ECD-39, ECD-45, ECD-49, and RBD-21 scFv or IgG are found to separately bind to at least three different epitopes.

### Example 6 Binding to Antigens Expressed in Mammalian Cells

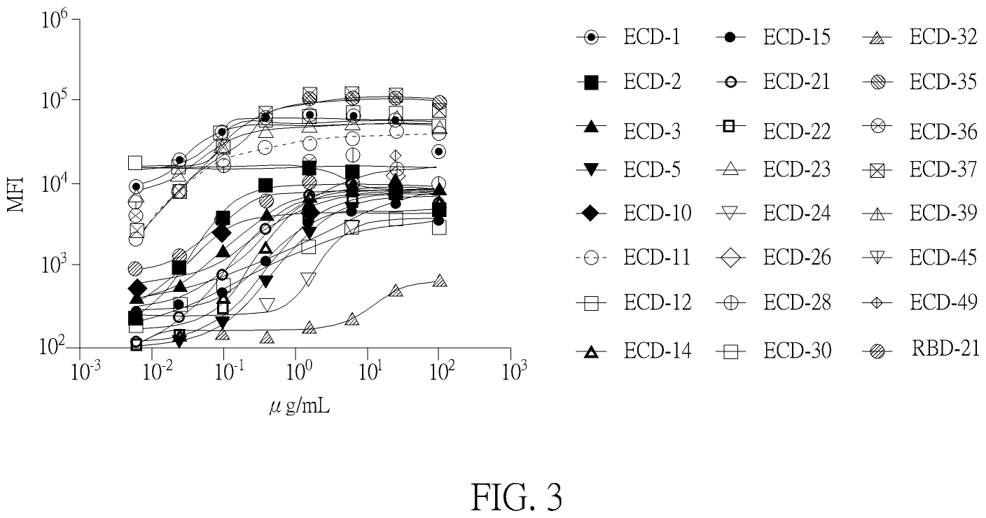

To check whether the IgGs bind to the correct conformation of S protein, the binding ability of ECD-1, ECD-2, ECD-3, ECD-5, ECD-10, ECD-11, ECD-12, ECD-14, ECD-21, ECD-22, ECD-24, ECD-26, ECD-28, ECD-30, ECD-35, ECD-36, ECD-37, ECD-39, ECD-45, ECD-49, and RBD-21 IgG toward S protein on cell surface was confirmed by flow cytometry. Plasmids encoding S protein were transfected into CHO cells. Above IgGs were serial diluted with PBS buffer, and added to S protein-expressed cell. Signals were detected with fluorescence labelled anti-human IgG antibody by flow cytometry. The results are shown in FIG. 3. MFI is mean fluorescence intensity, representing the amount of IgGs binding to the S protein.

The binding ability of ECDs and of RBD-21 toward S protein on cell surface are shown in FIG. 3. As shown in FIG. 3, ECD-1, ECD-2, ECD-3, ECD-5, ECD-10, ECD-11, ECD-12, ECD-14, ECD-21, ECD-22, ECD-24, ECD-26, ECD-28, ECD-30, ECD-35, ECD-36, ECD-37, ECD-39, ECD-45, ECD-49, and RBD-21 IgG antibodies exhibit various binding affinity to S protein expressed on cell surface at different concentrations.

### Example 7 ACE2 Competition

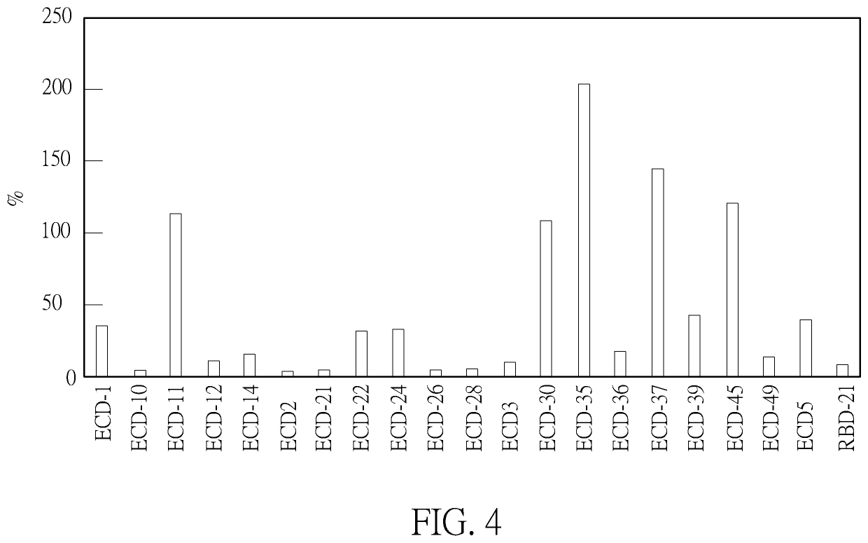

S protein was expressed on CHO cell surface (CHO-COVID-19-spike cell). 150 μg/ml antibody was added to CHO-COVID-19-spike cell on microplate, and then purified ACE2-8×His protein 10 μg/ml was added to each well. ACE2-8×His were detected with mouse anti-His antibody and anti-mouse-Fc (APC-conjugated). The ACE2 competition rates (%) are shown in FIG. 4.

FIG. 4 shows the ACE2 binding rate. As can be seen, ECD-1, ECD-2, ECD-3, ECD-5, ECD-10, ECD-11, ECD-12, ECD-14, ECD-21, ECD-22, ECD-24, ECD-26, ECD-28, ECD-30, ECD-35, ECD-36, ECD-37, ECD-39, ECD-45, ECD-49, and RBD-21 IgG competes with ACE2 and reduce the binding between ACE2 and S protein at different levels.

### Example 8 Binding Affinity Assay

The binding affinities of ECD-2, ECD-14, ECD-21, ECD-28, and ECD-36 IgG were measured by Biacore 8K (Cytiva). Antibodies were immobilized on the surface of CM5 chip, and different concentration of spike ECD trimer (1 nM-32 nM, two-fold serial dilution) were injected for 150 seconds at a flow rate of 50 μl/min with a 10 minutes of dissociation phase in HBS-EP running buffer. The kinetic parameters were obtained to a 1:1 binding model (Cytiva). The K_{D} values in Example 8 are shown in Table 1.

TABLE 1

| Ab ID | K_{D} (nM) |
|---|---|
| ECD-2 | 0.447 |
| ECD-14 | 4.160 |
| ECD-21 | 0.099 |
| ECD-28 | 0.100 |
| ECD-36 | 0.082 |

### Example 9 Binding Affinity Assay of SARS-CoV-2 B.1.351 Variant

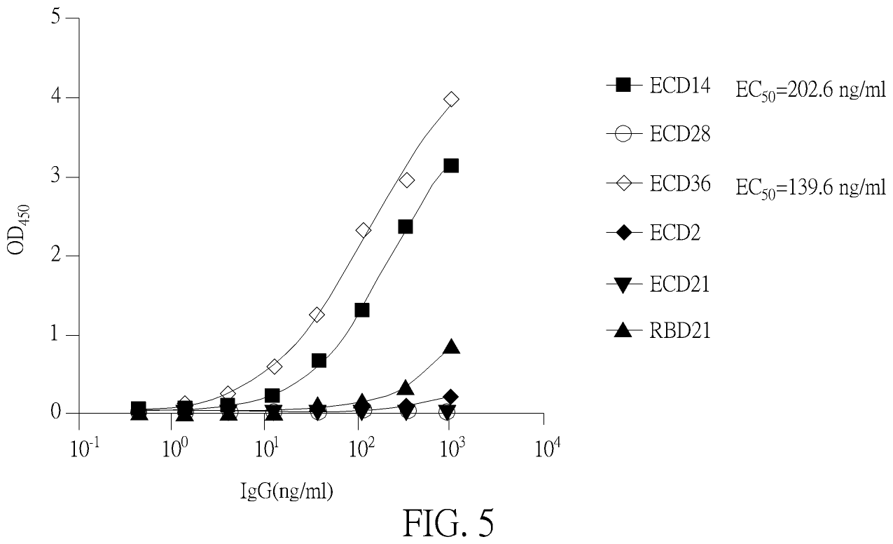

The binding affinities of ECD-2, ECD-14, ECD-21, ECD-28, ECD-36, and RBD-21 IgG toward S protein RBD of B.1.351 strain (SEQ ID NO: 46) were examined by ELISA. S protein RBD of B.1.351 strain purified from HEK293 cell was coated on ELISA plate at the concentration of 0.5 μg/well. After blocking with 5% skim milk, serial diluted IgG were added to each well. The signals were detected by anti-human antibody. The results are shown in FIG. 5.

The binding affinities of ECDs and RBD-21 toward S protein RBD of B.1.351 strain are shown in FIG. 5. As shown in FIG. 5, ECD-14, and ECD-36 remain binding affinities toward S protein of B.1.351 strain.

### Example 10 Pseudovirus Neutralization

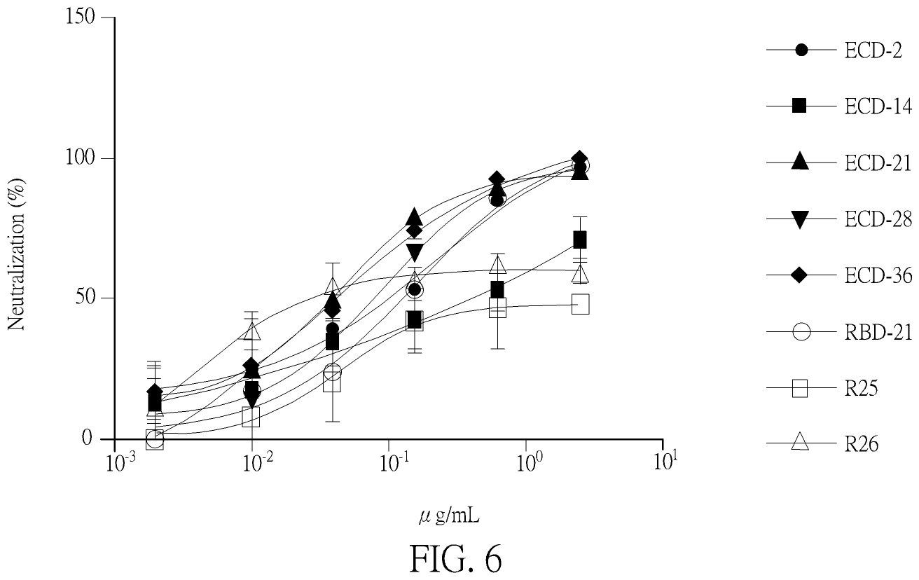

The SARS-CoV-2 pseudoviruses were produced by transfected with pCMVdeltaR8.91, pLAS3w.FLuc.puro and pcDNA3.4-SARS-CoV-2-Spike. After incubation with ECD-2, ECD-14, ECD-21, ECD-28, ECD-36, RBD-21 IgG and reference antibodies R25 and R26, the pseudoviruses were used to infect mammalian cells expressing ACE2. Luciferase activity was determined according to the instruction of Luciferase Assay System. The pseudovirus neutralization rate of the tested antibody was calculated based on the luciferase luminescence value. The reference antibodies R25 and R26 are recombinant antibodies constructed according to the variable domain sequences of antibodies derived from patients infected with SARS-CoV-2. The results are shown in FIG. 6.

FIG. 6 shows the pseudovirus neutralization rate of the tested antibodies. As can be seen in FIG. 6, ECD-2, ECD-21, ECD-28, and ECD-36 IgG exhibit nearly 100% reduction abilities at concentrations higher than 1 μg/mL.

### Example 11 SARS-CoV-2 B.1.351 Variant Pseudovirus Neutralization

The SARS-CoV-2 B.1.351 pseudoviruses were purchased from Academia Sinica RNA Technology Platform and Gene Manipulcation Core. The SARS-CoV-2 B.1.1.7 pseudoviruses were produced by transfected with pCMVdeltaR8.91, pLAS3w.FLuc.puro and pcDNA3.4-SARS-CoV-2-Spike-B.1.1.7. pcDNA3.4-SARS-CoV-2-Spike-B.1.1.7 encoding the amino acid sequence (SEQ ID NO: 47). After incubation with ECD-36 and reference antibodies R30, the pseudoviruses were used to infect mammalian cells expressing ACE2. Luciferase activity was determined according to the instruction of Luciferase Assay System. The pseudovirus neutralization rate of the tested antibody was calculated based on the luciferase luminescence value. R30 is a neutralizing antibody developed by Eli Lilly (LY-CoV555) and is reconstructed in our laboratory. The results are shown in Table 2.

As can be seen, R30 losses neutralization ability against B.1.351. On the other hand, ECD-36 remains the ability to neutralize B.1.351.

TABLE 2
IC₅₀ (ng/mL) of pseudovirus neutralization

| IgG | Alpha | Beta |
|---|---|---|
| | B.1.1.7 | B.1.351 |
| R30 | 58.2 | ND |
| ECD-36 | 165.3 | 96.21 |

### Example 12 True Virus Neutralizing Assay

The PRNT (Plaque reduction neutralization tests) was performed in triplicate using 24-well tissue culture plates (TPP Techno Plastic Products AG, Trasadingen, Switzerland) in a biosafety level 3 facility with ECD-2, ECD-14, ECD-21, ECD-28, and ECD-36 IgG (Ab ID). Serial dilutions of serum samples were incubated with 30-40 plaque-forming units of virus for 1 h at 37° C. The virus-serum mixtures were added onto Vero E6 cell monolayers and incubated for 1 hr at 37° C. in 5% CO2 incubator. Then the plates were overlaid with 1% agarose in cell culture medium and incubated for 3 days when the plates were fixed and stained. Cells were washed once with PBS, and supplemented with 1.2% microcrystalline cellulose solution in Dulbecco modified Eagle medium. After 3 days, the samples were fixed and inactivated by 6% formaldehyde/PBS solution and stained with crystal violet. IC₅₀ were estimated from the reduction in the number of plaques. The results are shown in Table 3

TABLE 3
True Virus Neutralization

| Ab ID | IC₅₀ (μg/ml) |
|---|---|
| ECD-36 | 0.266 ± 0.084 |
| ECD-21 | 0.346 ± 0.111 |
| ECD-28 | 0.397 ± 0.088 |
| ECD-2 | 0.770 ± 0.086 |
| ECD-14 | >1.0 |

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A SARS-CoV-2 binding molecule specifically binding to SARS-CoV-2 S protein, comprising:
(a) a light chain CDR1, a light chain CDR2 and a light chain CDR3 respectively comprising the amino acid sequences of SEQ ID NO: 14, 15, and 16, and a heavy chain CDR1, a heavy chain CDR2 and a heavy chain CDR3 respectively comprising the amino acid sequences of SEQ ID NO: 30, 31, and 32;
(b) a light chain CDR1, a light chain CDR2 and a light chain CDR3 respectively comprising the amino acid sequences of SEQ ID NO: 17, 18, and 19, and a heavy chain CDR1, a heavy chain CDR2 and a heavy chain CDR3 respectively comprising the amino acid sequences of SEQ ID NO: 33, 34, and 35;
(c) a light chain CDR1, a light chain CDR2 and a light chain CDR3 respectively comprising the amino acid sequences of SEQ ID NO: 20, 21, and 22, and a heavy chain CDR1, a heavy chain CDR2 and a heavy chain CDR3 respectively comprising the amino acid sequences of SEQ ID NO: 36, 37, and 38;
(d) a light chain CDR1, a light chain CDR2 and a light chain CDR3 respectively comprising the amino acid sequences of SEQ ID NO: 23, 24, and 25, and a heavy chain CDR1, a heavy chain CDR2 and a heavy chain CDR3 respectively comprising the amino acid sequences of SEQ ID NO: 39, 40, and 41; or
(e) a light chain CDR1, a light chain CDR2 and a light chain CDR3 respectively comprising the amino acid sequences of SEQ ID NO: 26, 27, and 28, and a heavy chain CDR1, a heavy chain CDR2 and a heavy chain CDR3 respectively comprising the amino acid sequences of SEQ ID NO: 42, 43, and 44.

2. The SARS-CoV-2 binding molecule of claim 1, wherein the binding molecule comprises a light chain variable domain (VL) and a heavy chain variable domain (VH) that are at least 80% identical in amino acid sequence to the VL domain selected from the group consisting of SEQ ID NO: 4, 6, 8, 10 and 12, and a VH domain selected from the group consisting of SEQ ID NO: 5, 7, 9, 11, and 13.

3. The SARS-CoV-2 binding molecule of claim 1, wherein the binding molecule is a recombinant antibody thereof.

4. The SARS-CoV-2 binding molecule of claim 3, wherein the binding molecule is an IgG or a scFv.

5. The SARS-CoV-2 binding molecule of claim 4, wherein the binding molecule comprises a light chain constant domain of the amino acid sequence of SEQ ID NO:29, and a heavy chain constant domain of the amino acid sequence of SEQ ID NO:45.

6. The SARS-CoV-2 binding molecule of claim 1, wherein the binding molecule is a multispecific antibody or a multispecific antibody fragment.

7. A cell comprising a nucleic acid, wherein the nucleic acid comprises a sequence encoding the binding molecule of claim 1.

8. A composition comprising at least one of a first binding molecule SARS-CoV-2 and a second binding molecule, wherein the first binding molecule and the second binding molecule are anyone of the SARS-CoV-2 binding molecule of claim 1, wherein the first binding molecule is different from the second binding molecule.

9. The composition of claim 8, further comprising an anti-viral agent.

10. The composition of claim 9, wherein the first binding molecule comprises a light chain CDR1, a light chain CDR2 and a light chain CDR3 respectively comprising the amino acid sequences of SEQ ID NO: 26, 27, and 28, and a heavy chain CDR1, a heavy chain CDR2 and a heavy chain CDR3 respectively comprising the amino acid sequences of SEQ ID NO: 42, 43, and 44.

11. The composition of claim 9, wherein the anti-viral agent is an antibody specifically binds to a different epitope from the first binding molecule on the SARS-CoV-2 S protein.

12. The composition of claim 10, wherein the anti-viral agent is an antibody specifically binds to a different epitope from the first binding molecule on the SARS-CoV-2 S protein.

13. A method for decreasing S protein-mediated coronavirus binding to cells, comprising the step of contacting the coronavirus with the binding molecule according to claim 1.

14. The method of claim 13, wherein the coronavirus is SARS-CoV-2.

15. A method for treating, preventing, or alleviating the symptoms of a coronavirus-mediated disorder in a subject in need, comprising the step of administering to said subject the SARS-CoV-2 binding molecule of claim 1.

16. A method for treating, preventing, or alleviating the symptoms of a coronavirus-mediated disorder in a subject in need, comprising the step of administering to said subject the composition according to claim 8.

17. The method of claim 16, wherein the coronavirus-mediated disorder is COVID-19.
